(19) United States
(12) Patent Application Publication
(10) Pub. No.: US 2007/0298085 A1
(43) Pub. Date: Dec. 27, 2007

(54) Skin Sanitizing Object

(76) Inventors: David J. Lestage, Livermore, CA (US); Marc Privitera, Walnut Creek, CA (US); Scott Cumberland, Tracy, CA (US)

Correspondence Address: THE CLOROX COMPANY, P.O. BOX 24305, OAKLAND, CA, 94623-1305, US

(21) Appl. No.: 11/426,808

(22) Filed: Jun. 27, 2006

(51) Int. Cl. A61K 9/70

(52) U.S. Cl. 424/443

(57) ABSTRACT

The present invention provides a novel method of sanitizing skin surfaces, preferably the hands. The sanitizing object is a moldable and pliable composition with at least one regenerable surface and an antimicrobial agent incorporated therein. The antimicrobial agent is capable of transfer from the object to a skin surface. The antimicrobial agent is capable of regeneration upon manipulation of the object.

### FIELD OF THE INVENTION

The present invention relates to objects and methods of sanitizing a skin area. More specifically, the invention relates to a moldable and pliable composition used to sanitize a skin area, preferably the hand.

### BACKGROUND OF THE INVENTION

Hands are a part of the body most frequently exposed to microorganisms or germs (i.e., bacteria, viruses and fungi). Microorganisms are commonly spread from a person's contaminated hands to their eyes, mouth or nose. Microorganisms can also be spread by ingesting food handled with contaminated hands. Likewise, microorganisms can be quickly passed from individual to individual through hands and, thus, contribute to the spread of contagious or infectious diseases.

Hand washing with soap and water is the simplest and one of the most effective ways to keep from becoming infected with microorganisms or giving them to others. However, washing with soap and water is not always available or convenient. Thus, many people use hand sanitizers in the absence of soap and water. Hand sanitizing products sanitize skin surfaces without the need for water and/or towels.

Hand sanitizing products currently available today include wipes, sprays, gels and foams which require some type of applicator to use. Applicators include, for example, squeeze bottles, finger pump sprayer or roamer, and wipes. Other devices useful to facilitate quick access to these hand sanitizing products are also known, such as described in US Patent Publication No. 2006/0078484 which describes a wearable hand sanitizing device structured to attach to the body or an item of clothing so that the source of antimicrobial agent is readily accessible. Also known is the SPRIXX® dispenser, engineered to be worn on the body, operate using one hand, and at your side for quick access when a hand sanitizer is needed.

There exists a need for a hand sanitizing object that is fun, easy and convenient to use without a dispenser. For example, on-the-go and in a professional or public setting such as a school, grocery store, eating establishment, daycare, hospital, office and the like. There is also a need for such an object that is easy to use by people with, for example, arthritis, the elderly and children.

Various play material compositions have been proposed through the years. Compositions of different types have been provided to amuse children and adults and to aid in the development of manual skills and dexterity as well as creativity. The variety of compositions is virtually endless, and includes the use of soft, pliable or moldable materials. Optionally additives are included to modify the characteristics of the composition.

McVicker et al., U.S. Pat. No. 3,167,440, describes a plastic modeling composition of a soft pliable, working consistency for being molded into any desired shape or form and is slow drying so as to be retained in a workable moldable condition for a long period of time. The composition can be repeatedly reworked and molded into different shapes and forms. The McVicker patent describes incorporation of borax, salicylic acid, sodium benzoate, sorbic acid, sodium and calcium propionate, calcium oxide, colemanite, resorite, and kernite. These materials are described to function as a drying agent and as such they have an antiseptic effect upon the composition in that they inhibit or prohibit the growth and activity of microorganisms to preserve the desirable properties of the composition. The McVicker patent issued in 1965 for the well known children's toy PLAYDOH®.

Wright, U.S. Pat. No. 2,541,851, describes compositions comprising dimethyl silicone with a compound of boron, preferably followed by a further treatment of the product with heat, a catalyst, or both. The Wright patent issued in 1951 for the well known children's toy SILLY PUTTY®.

Desbiens, U.S. Pat. No. 5,538,551, describes a modeling composition including a food grade anti-mold agent, such as sodium benzoate. Also described is incorporation of essential oils for enhanced fragrance. Abimael, U.S. Pat. No. 5,972,092, describes lightweight flour based modeling dough composition of matter having unique pliability and moldability. Also described are preservatives used to prevent microbial growth, salt stabilizers, fragrances, and colorants.

Accordingly, various agents have been described for use in moldable compositions for the purpose of inhibiting microbial growth within the compositions.

Various antimicrobial skin sanitizers have been proposed through the years. Most products are aqueous or non-aqueous and must be used with an appropriate dispenser, such as a wipe, pump, spray or squeeze bottle.

The present invention provides a novel method of sanitizing skin surfaces without the need for soap and water. One aspect of the present invention is directed to a moldable composition including an antimicrobial agent used to sanitize a skin area. The composition has a plurality of surfaces and upon manipulation of the composition the antimicrobial agent is activated on one or more surfaces and transferred to a skin area.

These and other aspects will become readily apparent from the detailed description which follows.

### BRIEF DESCRIPTION OF THE DRAWINGS

FIG. 1 shows microbial inhibition in the sanitizing object on Petri dishes.

FIG. 2 shows a graph of microbial inhibition on gloves and in a sanitizing object.

FIG. 3 shows a graph of residual microbial efficacy

### SUMMARY OF THE INVENTION

In one aspect, the invention relates to a sanitizing object, comprising a moldable and pliable solid composition, wherein the object is capable of being manipulated by a hand, a plurality of surfaces capable of regeneration upon manipulation, an antimicrobial agent distributed within the composition and transferred from one or more surfaces of the object to a skin area, wherein the antimicrobial agent is regenerated on the surface.

In another aspect, the antimicrobial agent is transferred during manipulation of the object. In yet another aspect, the skin area is a hand.

In another aspect, the sanitizing object is comprised of flour, silicone polymer, wax, clay or a polysaccaride.

Another aspect of the present invention is to instantly sanitize a skin area without the need for soap and water. In yet another aspect of the invention, the skin sanitizing object is safe and effective to kill bacteria for two or more uses.

Yet another aspect of the invention is a hand sanitizing object that includes a color change indicator for functional or aesthetic purposes.

In another aspect, the invention relates to a sanitizing object, comprising a moldable and pliable solid composition having a rupture time value measured by the method specified of from about 10 seconds to about 18 seconds.

The present invention further relates to methods for using the skin sanitizing object and a kit for preparing the object.

### DETAILED DESCRIPTION OF THE INVENTION

The skin sanitizing objects of the present invention can comprise, consist of or consist essentially of the essential elements and limitations of the invention described herein, as well any of the additional or optional ingredients, components, or limitations described herein.

Before describing the present invention in detail, it is to be understood that this invention is not limited to particularly exemplified objects, methods or kits that may, of course, vary. It is also to be understood that the terminology used herein is for the purpose of describing particular embodiments of the invention only, and is not intended to limit the spirit and scope of the invention in any manner.

All publications, patents and patent applications cited herein, whether supra or infra, are hereby incorporated by reference in their entirety to the same extent as if each individual publication, patent or patent application was specifically and individually indicated to be incorporated by reference.

### Definitions

“Antibacterial agent” or “Antimicrobial agent” as used herein includes agents capable of killing, inhibiting or reducing the growth of any of a broad spectrum of pathogenic microorganisms such as bacteria, yeast, fungi, algae, viruses, and mold.

“Instant Sanitizing” as used herein means the sanitizing object of the present invention sanitizes a skin area within 30 seconds without the need for soap and water.

“Moldable” as used herein includes a composition that is deformable, such that it changes shape, size or position as a result of force, and retains its shape after the force is removed.

“Pliable” as used herein includes a soft composition capable of bending freely or repeatedly without breaking.

“Rupture Test” as used herein includes a method of measuring the deformation of a composition by force, which eventually results in rupture of the composition. Rupture is influenced by the speed by which the force is applied and the size of the sample.

“Sanitize” as used herein means that any of a broad spectrum of pathogenic microorganisms such as bacteria, yeast, fungi, algae, viruses, and mold is killed, inhibited or reduced. The sanitizing activities of the object of the present invention are readily regenerable upon manipulation.

As used herein and in the claims, the term “comprising” is inclusive or open-ended and does not exclude additional unrecited elements, compositional components, or method steps. Accordingly, the term “comprising” encompasses the more restrictive terms “consisting essentially of” and “consisting of”.

It must be noted that, as used in this specification and the appended claims, the singular forms “a,” “an” and “the” include plural referents unless the content clearly dictates otherwise. Thus, for example, reference to a “surfactant” includes two or more such surfactants.

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

The skin sanitizing object of the present invention is described in detail hereafter.

### PREFERRED EMBODIMENTS

Composition of Object

The composition of the sanitizing object of the present invention includes the use of any soft, pliable or moldable material, which is non-toxic, safe for handling, and safe for application to a skin area. Optionally the object may be made of a combination of materials and additives intended to modify the characteristics of the composition.

In one aspect, the composition comprises flour, which is described as a fine powder made from cereals or other starchy food sources. It is most commonly made from wheat, but also maize (a.k.a. corn), rye, barley, and rice, amongst many other grasses and non-grain plants (including many Australian species of acacia). Flour can also be made from legumes and nuts, such as soy, peanuts, almonds, and other tree nuts. A non-limiting example of an object comprising flour is PLAYDOH®.

In yet another aspect, the composition is comprised of a polysaccharide material, for example, glycans, which is described as a complex carbohydrate or polymer made up of many monosaccharides joined together by glycoside linkages. Non-limiting examples include starch, glycogen, cellulose and chitin. Additional polysaccharides include gums such as alginates, xanthates, pectins, carrageenans, guar, carboxymethyl cellulose, and the like.

In yet another aspect, the composition is comprised of silicones or polysiloxanes which are described as inorganic polymers consisting of a silicon-oxygen backbone with side groups attached to the silicon atoms. Non-limiting examples include dimethyl silicone polymers, such as SILLY PUTTY®, heat vulcanizable silicone polymers, such as vinyl-substituted dimethyl silicones and phenyl-substituted dimethyl silicones. Other examples include organo-silicon compounds which may be polymerized to high molecular weight compositions by treating with boric oxide are the hydrolysis products of methylethyl silicon dichloride, diethyl silicon dichloride, dipropyl silicon dichloride, diethyldiethoxysilicane, dibutyldiethoxysilicane, diamyl silicon dichloride, methyl silicon trichloride, ethyltriethoxysilicane, propyl silicon trichloride, phenyl silicon trichloride, phenylethyl silicon dichloride, phenylmethyldiethoxysilicane, diphenyl silicon dichloride.

In another aspect, the composition is comprised of a wax type material, which is described as a malleable solid with a melting point of 45° C. or above consisting of hydrophobic water insoluble moieties generated through natural or synthetic processes. Non-limiting examples include animal, insect, vegetable, mineral, petroleum, and synthetic waxes. Non-limiting examples of animal wax would be lanolin (wool wax) and spermaceti (sperm whale). Insect varieties would include beeswax and shellac wax. Vegetable waxes would include bayberry, carnauba, castor, rice bran and the like. Mineral types may be derived from coal or lignite beds such as montan or ozocerite waxes. Petroleum waxes would be those consisting of long chain hydrocarbons such as paraffin whereas synthetic varieties would be based on but not limited to polyethylenes.

In another aspect, the composition is comprised of a clay type material, which is described as a group of hydrous aluminium phyllosilicates minerals. Clay consists of a variety of phyllosilicate minerals rich in silicon and aluminium oxides and hydroxides which include variable amounts of structural water. Non-limiting examples include PLASTICINE®, earthy clays, polymer clays (based on the synthetic polymer polyvinyl chloride), paper clays and hydrate clays which would include clays reinforced with wood pulp, wire mesh, polymers such as nylon, polystyrene, and the like.

In another aspect, the moldable material may be made of an elastomeric amorphous polymer, for example, a thermoset or thermoplastic material above the glass transition temperature so that considerable segmental motion is possible. Non-limiting examples include natural rubber, polyisoprene, butyl rubber, polybutadiene, styrene-butadiene, nitrile rubber (acrylonitrile, alpha-chloroacrylonitrile, alpha-fluoroacrylonitrile, methacrylonitrile, ethacrylonitrile and the like), chloroprene, polychoroprene, polyurethane rubber, fluorosilicone rubber, poly(vinyl naphthalene), poly(vinyl pyridine), polyacrylic, polysulfide, chlorosulfonated polyethylene, ethylene-propylene rubber, and mixtures and copolymers thereof.

The level is selected to provide the desired moldable and pliable characteristics and can be modified as desired. Preferred materials include flour such as, PLAYDOH®, available from Hasbro™; a silicone polymer, such as SILLY PUTTY®, available from Binney and Smith; and PLASTICINE®.

Antimicrobial Agents

In one embodiment, the antimicrobial agent is dispersed homogeneously throughout the object. In another embodiment, the antimicrobial agent is bound or entrained within the object.

Non-limiting examples of antimicrobial agents include:

Phenols: Triclosan, PCMX, and the like;

Biguanides: PHMB, CHG, and the like;

Metals: Metal salts, which generally includes salts of metals in groups 3b-7b, 8 and 3a-5a. Specifically are the salts of aluminum, zirconium, zinc, silver, gold, copper, lanthanum, tin, mercury, bismuth, selenium, strontium, scandium, yttrium, cerium, praseodymium, neodymium, promethum, samarium, europium, gadolinium, terbium, dysprosium, holmium, erbium, thulium, ytterbium, lutetium and mixtures thereof;

Quats: Ammonium salts like benzalkonium chloride, benzethonium chloride and cetrimide, and the like;

Acids: Lactic, Citric, Glycolic, Organic/Inorganic Acids, and the like;

Alkaline Agents: Ca(OH)₂, and the like;

Dyes: Non-limiting dyes include, gentian, or crystal violet, ethyl violet, brilliant green, etc., and the FD&C dyes such as Blue No. 1 and Green No. 3. In addition, other dyes include the following FD&C and D&C colors: (1) Monoazo dyes such as, but not limited to, FD&C Yellow No. 5, FD&C Yellow No. 6, (2) Diazo dyes such as, but not limited to, D&C Red No. 17, (3) Indigoid dyes such as, but not limited to, FD&C Blue No. 2, (4) Xanthene (Fluorescein) dyes such as, but not limited to, FD&C Red No. 3, (5) Anthraquinone dyes such as, but not limited to, D&C Green No. 6, (6) Quinoline dyes such as, but not limited to, D&C Yellow No. 1.

Halogens: NaOCl, Ca(OCl)₂, ClO₂, and the like;

Inorganic oxides/hydroxides: Insoluble inorganic oxides with isoelectric points greater than the pH of the solution have been shown to be efficient at the physical removal of microorganisms (bacteria and virus). Non-limiting examples include magnesium hydroxide, magnesium oxide, aluminum oxide, iron oxide, cerium oxide, zinc oxide, zirconium oxide, barium oxide, calcium oxide, hydroxyapatite, chromium oxide, cobalt oxide, cesium oxide, and chrysotile asbestos. The antimicrobial capability of these materials can be improved by doping with antimicrobial metals such as silver and the like;

Oxidants: H₂0₂, Perborate, and the Like;

Naturals: Also useful as antimicrobial agents are referred to as “natural essential oils”. These actives derive their names from their natural occurrence in plants. Typical natural essential oil antibacterial actives include oils of anise, lemon, orange, rosemary, wintergreen, thyme, lavender, cloves, hops, tea tree, citronella, wheat, barley, lemongrass, cedar leaf, cedarwood, cinnamon, fleagrass, geranium, sandalwood, violet, cranberry, eucalyptus, vervain, peppermint, gum benzoin, basil, fennel, fir, balsam, menthol, ocmea origanum, Hydastis carradensis, Berberidaceae daceae, Ratanhiae and Curcuma longa. Also included in this class of natural essential oils are the key chemical components of the plant oils which include, but are not limited to, anethol, catechole, camphene, carvacol, eugenol, eucalyptol, ferulic acid, famesol, hinokitiol, tropolone, limonene, menthol, methyl salicylate, thymol, terpineol, verbenone, berberine, ratanhiae extract, caryophellene oxide, citronellic acid, curcumin, nerolidol and geraniol.

Preferred antimicrobial agents include natural actives, such as d-limonene available from Firmenich (New Jersey, USA), Lavender oil available from Firmenich (New Jersey, USA), Tea tree oil, available from commercial Brand Now Foods; and mixtures thereof. Also preferred is Lactic Acid, available from Purac (Lincolnshire, Ill.); quaternary ammonium compounds available from Lonza (Switzerland); silver di-hydrogen citrate available from Ciba Specialty Chemicals (Tarrytown, N.Y.), inorganic silver antimicrobials from AirQual and mixtures thereof.

The antimicrobial agents useful in the present invention can be present at a level of from about 0.01% to about 10% by weight of the composition, and preferably from about 0.1% to about 5% by weight. The level is selected to provide the desired level of antimicrobial activity and can be modified as desired.

Indicators

In one aspect, the indicator provides a clear and obvious visual or physical indication to the user that the antimicrobial agent is working, has been depleted, is no longer working or that the sanitizing object is overloaded with microorganisms. In another aspect, the indicator is used as an aesthetic enhancement.

In one embodiment, the indicator is dispersed homogeneously throughout the object. In another embodiment, the indicator is bound or entrained within the object. In another embodiment, the indicator is encapsulated.

In another embodiment, the indicator is a physical degradation of the object. Degradation may be based on the incorporation of a material, which breaks down over a specified period of time. A non-limiting example includes Polyvinyl alcohol (PVOH) crosslinked with glycerine in an aqueous solution. When the PVOH dries out, the composition falls apart or degrades.

In yet another embodiment the indicator is a visual color change experience. In one aspect the color change is based on a change in pH. In another aspect, the color change is based on an indicator dye, capable of generating a visible color change within a relevant pH range or reducing environment. Examples of relevant pH change indicator dyes are listed in Table 1 below.

TABLE 1
pH Change Indicators

| Range of visible | | | |
|---|---|---|---|
| color change | Indicator | Color acidic | Color basic |
| 0.2–1.8 | Cresol red | Red | Yellow |
| 1.2–2.8 | m-Cresol purple | Red | Yellow |
| 1.2–2.8 | Thymol blue | Red | Yellow |
| 2.0–4.4 | 2,6-Dinitrophenol | Colorless | Yellow |
| 2.8–4.7 | 2,4-Dinitrophenol | Colorless | Yellow |
| 2.9–4.0 | 4-Dimethyl | Red | Orange |
| | aminoazobenzene | | |
| 3.0–4.6 | Bromophenol blue | Yellow | Purple |
| 3.0–5.2 | Congo red | Blue | Red |
| 3.1–4.4 | Methyl Orange | Red | Yellow |
| 3.5–5.5 | Ethoxychrysodine | Red | Yellow |
| | hydrochloride | | |
| 3.6–5.4 | Bromocresol green | Yellow | Blue |
| 4.0–5.8 | 2,5-Dinitrophenol | Colorless | Yellow |
| 4.4–6.2 | Methylene red | Red | Yellow |
| 4.8–6.4 | Chlorphenol red | Yellow | Purple |
| 5.2–6.8 | Bromocresol purple | Yellow | Purple |
| 5.4–7.5 | 4-Nitrophenol | Colorless | Yellow |
| 5.7–7.4 | Bromoxylenol blue | Yellow | Blue |
| 6.0–7.6 | Bromothymol blue | Yellow | Blue |
| 6.4–8.2 | Phenol red | Yellow | Red |
| 6.6–8.6 | 3-Nitrophenol | Colorless | Orange |
| 6.8–8.0 | Neutral red | Bluish red | Orange |
| 7.0–8.8 | Cresol red | Yellow | Purple |
| 7.3–8.7 | 1-Napthol phthalein | Brown | Blue |
| 7.4–9.0 | m-Cresol purple | Yellow | Purple |
| 8.0–9.6 | Thymol blue | Yellow | Blue |
| 8.2–9.8 | Phenolphthalein | Colorless | Violet |
| 9.3–10.5 | Thymolphthalein | Colorless | Blue |
| 10.0–12.1 | Alizarin yellow | Yellow | Brownish yellow |
| 11.6–13.0 | Epsilon blue | Orange | Violet |

In another embodiment, the pH color change indicator is an Anthocyanin, which is a water soluble vacuolar flavonoid pigment that reflects in the red to blue range of the visible spectrum depending on the pH of the surrounding solution. An Anthocyanin appears red in acidic solutions and blue in bases.

In another embodiment, color change is based on pressure or temperature, such as a thermo-chromatic change.

In another embodiment, the color change is based on oxidation-reduction reactions. A non-limiting example of an oxidation-reduction indicator includes Resazurin (Alamar Blue), which is colorless in a reduced state and pink in an oxidized state.

Other known methods of color change known in the art may be employed accordingly.

Surfactants

The surfactant may be incorporated to enhance the property which causes surfaces to adhere to each other. A surfactant operative herein is a water soluble or water dispersible nonionic, anionic, cationic, or an amphoteric compound with emulsifying abilities. A surfactant operative herein is any conventional surfactant known to the art. A representative listing of surfactants and properties thereof is detailed in Remington's Pharmaceutical Sciences, 17^{th} edition (Mack Publishing Company).

Plasticizer

The plasticizer is a substance or material incorporated in a material to increase its flexibility, workability or extensibility. The level is selected to provide the desired level of moldability and pliability and can be modified as desired. Non-limiting examples include dialkyl phthalates and other suitable organic esters.

Humectant

The humectant is a substance to prevent the loss of water to any significant extent so that the user may reconstitute the mass to its original softness merely by wetting one's hands with water. Non-limiting examples include triglycerol, glycerin, propylene glycols, poly (ethylene glycols) and diethylene glycol.

Preservatives

The preservative is a substance added to increase shelf life and inhibit growth of microorganisms within the object. A wide variety of such materials is available commercially. Non-limiting examples include parabens, borax, and salts such as sodium carbonate, sodium chloride, and aluminum sulfate.

Other Ingredients

The sanitizing object may be made of a combination of materials and additives intended to modify the characteristics of the composition. The compositions of the present invention can comprise a wide range of optional ingredients. Non limiting examples include, skin sensates, such as menthol, fragrance components, sunscreen agents, insect repellant, vitamins, and the like.

### ALTERNATIVE EMBODIMENTS

Alternatively, the sanitizing object is described as a hand exerciser which can also be used to relieve stress. Office areas are well known to contain microorganisms and hand exercise or stress balls are frequently used at the office to relieve stress and/or exercise the hand after typing or writing for a long period of time.

In one embodiment, the hand exercise or stress ball is made of any soft, pliable or moldable material known in the art. In another embodiment, the core is soft and pliable while the surface can be a fabric or elastic covering. The hand exercise or stress ball optionally contains an antimicrobial agent that is controllably released upon manipulation or compression thereby sanitizing the hands.

The sanitization can be accomplished by a variety of mechanisms or combination of mechanisms. In one embodiment, the covering of the ball can be constructed to contain an antimicrobial agent. The microorganisms are removed from the surface of the hands by contact with the plurality of surfaces of the hand exercise or stress ball. In another embodiment, the antimicrobial agent is added to the interior of the ball and contained in a semi-permeable elastic membrane that controls the diffusion of the antimicrobial agent out of the device by the action of squeezing or manipulating the device. Such an antimicrobial can be a liquid, a solid that sublimes or dissolves, or a combination thereof.

In an alternative embodiment, the sanitizing object is attached to a rigid or flexible base and used accordingly. Non-limiting examples of a base are textiles (woven and non-woven), handles, strings, eraser pad, films, foils, fabrics, plastics, papers, metals, and alloys.

Methods for Sanitizing the Skin

The skin sanitizing object of the present invention is useful for sanitizing the skin when soap and water are not available or convenient to use. Generally, the skin sanitizing process involves manipulating the moldable composition to bring the active to the object surface, contacting the object with a skin area to transfer the antimicrobial active to the skin, wherein the skin is sanitized. The antimicrobial active can be regenerated upon further manipulation of the object.

The amount of the agent applied, the frequency of application and the period of use will vary widely depending upon the level of sanitization desired, e.g., the degree of microbial contamination.

Preferably, the skin sanitizing compositions of the present invention are used to sanitize human and/or animal hands and/or feet.

Kit for Preparing a Skin Sanitizing Object

A kit for making a hand sanitizing object may include a moldable and pliable solid composition and an antimicrobial agent for incorporation in the composition. Directions for use and application may optionally be included.

### EXAMPLES

The skin sanitizing objects illustrated in Examples 1-8 illustrate specific embodiments of the objects of the present invention, but are not intended to be limiting thereof. Other modifications can be undertaken by the skilled artisan without departing from the spirit and scope of this invention

All exemplified compositions can be prepared by conventional formulation and mixing techniques. Component amounts are listed as weight percents and exclude minor materials such as fillers, pigments, emulsifying agents, talc, etc., typically used to modify the compositions characteristics.

### Example 1

### Preparation of a Hand Sanitizing Object

52.13 grams of uncontaminated commercially available PLAYDOH® (Hasbro Inc.) was kneaded with gloved hands for 5 min. 2.5 grams of powdered Lactic acid (100% active—PURAC) was added to the PLAYDOH®. The composition was kneaded with gloved hands for an additional 5 min. The final composition comprises 95.43% modeling composition and 4.57% lactic acid.

### Example 2

### Preparation of a Hand Sanitizing Object

120 grams of all-purpose flour was added to 250 grams water and heated to 100° C. while stirring constantly. Upon boiling, the composition was removed from the heat and 28 grams of potassium bitartrate, 36 grams of sodium chloride and 10 grams of tea tree oil was added. Mixing was continued until the composition was cooled. The final composition comprises 27% flour, 56.3% water, 6.3% potassium bitartrate, 8.1% sodium chloride, and 2.3% tea tree oil.

### Example 3

### Preparation of a Hand Sanitizing Object

9.25 grams of a 4% sodium borate solution (Sigma-Aldrich) was combined with 1.5 grams of a silver-citrate solution (Ciba). To this, 99.25 grams of a 4% poly(vinyl alcohol) solution (Sigma-Aldrich) was slowly added while vortexing. The composition was allowed to mix for 3 minutes or until thick and allowed to set for an additional 5 min. without stirring. The final composition comprises 99.724% modeling composition and 0.276% silver-citrate.

### Example 4

### Method for Evaluation of Sanitizing Effect on a Surface

The ability of PLAYDOH® impregnated with an antimicrobial agent to disinfect a surface contaminated with a microorganism, S. aureus, was tested. Staphylococcus aureus (which is occasionally given the nickname golden staph) is a bacterium, frequently living on the skin or in the nose of a healthy person, that can cause illnesses ranging from minor skin infections (such as pimples, boils, and cellulitis) and abscesses, to life-threatening diseases such as pneumonia, meningitis, endocarditis, Toxic shock syndrome (TSS), and septicemia. This method can also be applied to test the ability of other antimicrobial agents to inhibit the growth of other bacteria.

The study method included testing the composition of Example 1 and the following steps:
An overnight culture of S. aureus was diluted 1:10 with Butterfield's buffer.
200 μl of the diluted suspension was added onto the surface of TSA agar plate, spread and allowed to absorb for 30 min.
Two 4 gram samples of PLAYDOH® (one with and one without Lactic acid) were weighed out and flattened to a 2-¼″ square to obtain a uniform surface area
Each flattened PLAYDOH® was placed on the surface of TSA plate with the lawn of bacteria, at room temperature.
After the 30 min contact time, patches of PLAYDOH® were aseptically removed and the plates were incubated overnight at 35° C.
Plates were examined for zone of inhibition

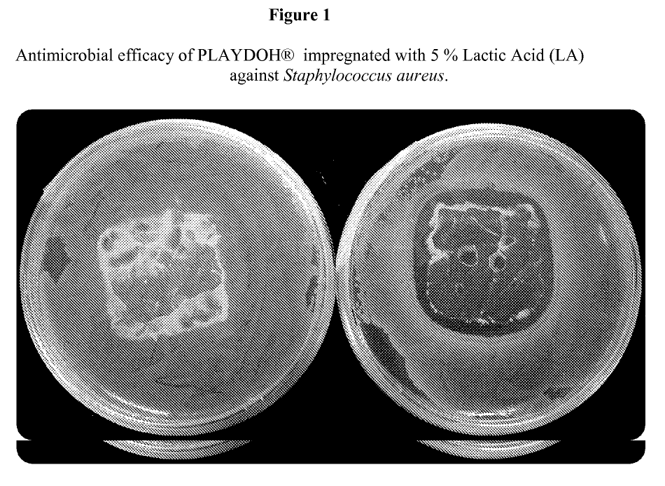

The results of the study method are shown in FIG. 1 and provide that PLAYDOH® with no antimicrobial agent (see left Petri dish of FIG. 1) shows confluent growth on the entire surface. In contrast, the plate that was in contact for 30 minutes with PLAYDOH® impregnated with Lactic Acid shows an obvious zone of inhibition around the contact site (see right Petri dish of FIG. 1) indicating that the PLAYDOH® impregnated with antimicrobial agent is able to prevent the growth of S. aureus on a contaminated surface.

### Example 5

### Method for Evaluation of Sanitizing Effect of Object on Hands

The ability of PLAYDOH® impregnated with Lactic acid was tested to determine the antimicrobial efficacy of the sanitizing object on hands and to determine the microbe transfer from hands to the sanitizing object. This method can also be applied to test the ability of other antimicrobial agents to inhibit the growth of other bacteria.

The study method included testing the composition of Example 1 and the following steps:
An overnight culture of S. aureus was diluted 1:10 with Butterfield's buffer.
4 grams of PLAYDOH® was weighed out and placed in sterile Petri dishes.
A sterile glove was placed on the left hand and the thumb and forefinger were inoculated with 50 μl (2×10⁵ CFU) of the diluted S. aureus culture.
The PLAYDOH® was then massaged with the contaminated fingers for 5 minutes and allowed to sit in the sterile Petri dish.
The contaminated fingers after massaging the PLAYDOH® were aseptically cut from the gloves and transferred in to a 10 ml tube of DE broth.
After 5 minutes, the PLAYDOH® was again massaged using a new sterile glove for another 5 minutes.
After 5 minutes of contact with the sterile glove, the PLAYDOH® was transferred into 10 ml tube of DE broth.
The fingers were again aseptically cut from the 2^{nd} glove and transferred into a 10 ml tube of DE broth
All the tubes were vortexed to extract bacteria from the glove and PLAYDOH® and enumerated on DE agar plates for viable bacteria counts.
The plates were incubated overnight at 35° C.

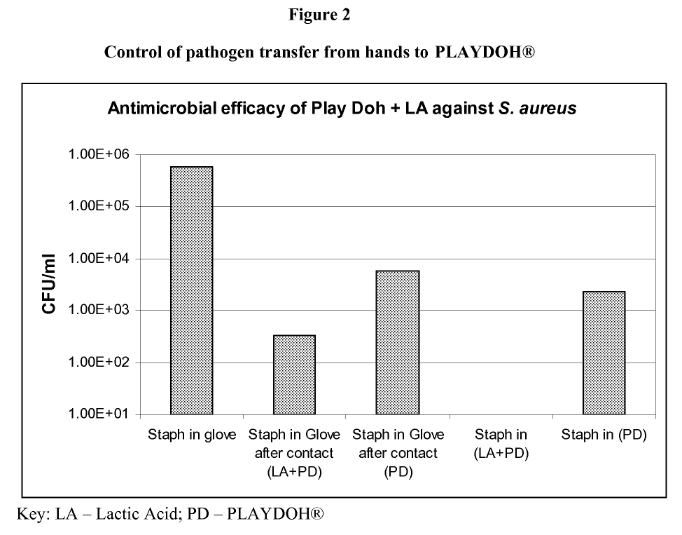

The results of the study method are shown in FIG. 2 and provide a noticeable reduction in S. aureus counts in gloves after massaging PLAYDOH® impregnated with Lactic acid for 5 minutes. An obvious difference in bacterial recovery from PLAYDOH® (with or without Lactic acid) after contact with contaminated gloves is also noticeable. No bacteria could be recovered from PLAYDOH® impregnated with Lactic acid, whereas the recovery of viable bacteria from PLAYDOH® with no actives is around 3 log units.

### Example 6

### Method for Evaluation of Residual Antimicrobial Effect of Sanitizing Object

The antimicrobial effect of the sanitizing object was tested. The composition of Example 1 and the method of Example 5 were used. This method can also be applied to test the ability of other antimicrobial agents to inhibit the growth of other bacteria.

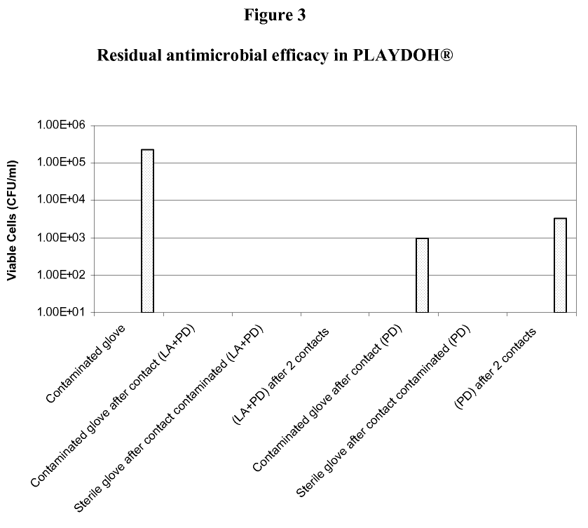

The results of the study method are shown in FIG. 3 and provide that bacteria could not be recovered from:
Contaminated glove after massaging PLAYDOH® impregnated with Lactic acid.
Sterile glove after massaging the contaminated PLAYDOH®impregnated with Lactic acid.
Sterile glove after massaging a contaminated PLAYDOH®.
PLAYDOH® impregnated with Lactic acid, including two exposures to a contaminated glove and two exposures to a sterile glove.
   FIG. 3 also provides that bacteria could be recovered from:
Contaminated glove after massaging PLAYDOH® with no antimicrobial agent.
PLAYDOH® with no antimicrobial agent, including two exposures to a contaminated glove and two exposures to a sterile glove.

### Example 7

### Method to Evaluate Rupture Point of Sanitizing Object

An INSTRON 5565/Q1216 was used to measure the rupture point of a sample of PLAYDOH®. Rupture is defined herein as the point of compression when the sample “cracks” or “splits” open. The top and bottom plates of the INSTRON were set to about 2.125 inches apart and the plate movement rate was set to 6.0 in/min. The room temperature was about 73.5° F. The rupture point of each sample was measured starting from the initial contact of the top compression plate with sample and the results are shown in Table 2.

TABLE 2
Rupture Point of PLAYDOH ®

| | Mass (g) PLAYDOH ® | Rupture Time (s) |
|---|---|---|
| 1 | 57.44 | 15.6 |
| 2 | 57.72 | 15.2 |
| 3 | 58.25 | 14.7 |
| 4 | 58.34 | 14.0 |
| 5 | 57.34 | 14.4 |
| 6 | 57.76 | 15.4 |
| 8 | 57.73 | 15.6 |

*Std Deviation: Time = 1.55 s

### Example 8

### Method to Evaluate Color Change Experience Based on pH

The ability of PLAYDOH® impregnated with Lactic acid to change color based on a pH change was tested. Bromophenol Blue powder, a color change indicator for the pH range of about 3.0-5.0 was used as the indicator. The indicator is yellow in acidic conditions (below pH 3.0) and purple in more basic conditions (above pH 4.6). The indictor may produce a blended color in the pH range 3.0-4.6.

First, 20-25 g of the composition prepared in Example 1, Composition 1, was tested. Using gloved hands, Composition 1 was flattened like a pancake and formed into a shallow bowl shape. 1-3 drops of water were added to the “bowl” and then 0.1-0.2 g of bromo blue powder was added. The water/bromo blue ingredients were massaged with gloved hands on the surface of Composition 1 until a pasty composition was formed. The pasty composition was then kneaded with gloved hands into Composition 1 for 1-3 minutes.

A second sample including 20-25 g of PLAYDOH® (and no antimicrobial agent) and 0.1-0.2 g sodium bicarbonate was prepared to test the indicator at a basic pH. The sodium bicarbonate was added to the pasty composition prepared by the method above and kneaded into the PLAYDOH® accordingly.

A third sample including PLAYDOH® (and no antimicrobial agent) and bromo blue was prepared by the method described above. The results of the three tests are shown in Table 3 below.

TABLE 3
pH Change Indicator Sample Results

| | Test Sample | Starting Color | End Color |
|---|---|---|---|
| 1 | PLAYDOH ® | White | Mustard Yellow |
| | Lactic Acid, and | | |
| | Bromo Blue | | |
| 2 | PLAYDOH ® | White | Dark Blue |
| | Sodium | | |
| | Bicarbonate | | |
| | Bromo Blue | | |
| 3 | PLAYDOH ®, | White | Light Blue/Blue |
| | Bromo Blue | | Green |
| 4 | PLAYDOH ® | White | White |

We claim:

1. A skin sanitizing object, comprising:
a) a moldable and pliable solid composition, wherein the object is capable of being manipulated by a hand;
b) a plurality of surfaces capable of regeneration upon manipulation;
c) an antimicrobial agent distributed within the composition and transferred from one or more surfaces of the object; and
wherein the antimicrobial agent is regenerated on the surface during manipulation.

2. The sanitizing object according to claim 1, wherein the antimicrobial agent is transferred during manipulation of the object.

3. The sanitizing object according to claim 1, wherein the skin area is a hand.

4. The sanitizing object according to claim 1, wherein the moldable and pliable solid composition is selected from the group consisting of flour, silicone polymer, wax, clay and a polysaccharide.

5. The sanitizing object according to claim 1, wherein the moldable and pliable solid composition is selected from the group consisting of flour or silicone polymer.

6. The sanitizing object according to claim 1, wherein the object can be used effectively for two or more uses.

7. The sanitizing object according to claim 1, wherein the antimicrobial agent is about 0.1 to about 10 weight percent of the object composition.

8. The sanitizing object according to claim 1, wherein the antimicrobial agent is selected from the group consisting of natural oils, lactic acid, quaternary ammonium compositions, silver di-hydrogen citrate and inorganic silver antimicrobials.

9. The sanitizing object according to claim 8, wherein the antimicrobial agent is selected from the group consisting of natural oils and lactic acid.

10. The sanitizing object according to claim 1, wherein the object further comprises an indicator.

11. The sanitizing object according to claim 10, wherein the indicator is capable of generating a visible color change within a relevant pH range.

12. The sanitizing object according to claim 1, wherein the composition has a rupture time measured by the method specified of from about 10 seconds to about 18 seconds.

13. The sanitizing object according to claim 1, wherein the object is a children's toy.

14. The sanitizing object according to claim 1, wherein the object is a hand stress or exercise ball.

15. The sanitizing object according to claim 1, wherein the object instantly sanitizes a skin area.

16. The sanitizing object according to claim 1, wherein the object further comprises a preservative.

17. A method of sanitizing skin, comprising:
a) manipulating a moldable and pliable composition comprising an antimicrobial agent;
b) contacting the composition with a skin area;
c) transferring the antimicrobial agent to the skin area;
d) manipulating the composition to regenerate the antimicrobial agent.

18. A method of delivering an antimicrobial agent to a skin area, wherein the antimicrobial agent is dispersed in a moldable and pliable hand sanitizing object, wherein the agent is transferred to the skin by contacting the object with the skin and wherein the antimicrobial agent is renewable by manipulation of the object.

19. A kit for making a hand sanitizing object comprising:
a) a moldable and pliable solid composition, wherein the object is capable of being manipulated by a hand; and
b) an antimicrobial agent for incorporation in the composition.
